(19) United States
(12) Patent Application Publication
(10) Pub. No.: US 2024/0247300 A1
(43) Pub. Date: Jul. 25, 2024

(54) A PLATFORM FOR THE FAST, LABEL-FREE, AUTOMATED EVALUATION OF STERILITY AND BIOBURDEN

(71) Applicant: The Texas A&M University System, College Station, TX (US)

(72) Inventors: Arum Han, College Station, TX (US); Paul J. de Figueiredo, College Station, TX (US)

(73) Assignee: The Texas A&M University System, College Station, TX (US)

(21) Appl. No.: 18/290,485

(22) PCT Filed: May 13, 2022
(86) PCT No.: PCT/US2022/029164

§ 371 Date: Nov. 14, 2023

Related U.S. Application Data
(60) Provisional application No. 63/188,741, filed on May 14, 2021.

(51) Int. Cl. C12Q 1/22; A61L 2/28; C12M 1/12; C12M 1/36; C12M 3/06

(52) U.S. Cl. CPC C12Q 1/22; A61L 2/28; C12M 23/16; C12M 37/06; C12M 41/48

(57) ABSTRACT

In an embodiment, the present disclosure pertains to a method for evaluation of sterility in a solution using impedance sensing. In another embodiment, the present disclosure pertains to a method for evaluation of bioburden in a solution. In a further embodiment, the present disclosure pertains to various devices for evaluation of sterility or bioburden.

### CROSS-REFERENCE TO RELATED APPLICATIONS

This patent application claims priority from, and incorporates by reference the entire disclosure of, U.S. Provisional Application 63/188,741 filed on May 14, 2021.

### TECHNICAL FIELD

The present disclosure relates generally to evaluation of sterility and bioburden and more particularly, but not by way of limitation, to a platform for the fast, label-free, automated evaluation of sterility and bioburden.

### BACKGROUND

This section provides background information to facilitate a better understanding of the various aspects of the disclosure. It should be understood that the statements in this section of this document are to be read in this light, and not as admissions of prior art.

Sterility refers to the non-appearance of viable microorganisms. Sterility testing is typically performed by taking a percentage of the total reagent or cellular inputs as well as the products to be tested in each manufactured batch. Conventional sterility testing typically relies on multi-day culture under different growth conditions, such as different growth media, to determine whether there are any viable microorganisms in the product being tested. These methods are time-consuming, costly, and are not amenable to in-line and/or continuous process monitoring.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method for evaluation of sterility in a solution using impedance sensing.

In another embodiment, the present disclosure pertains to a method for evaluation of bioburden in a solution.

In a further embodiment, the present disclosure pertains to various devices for evaluation of sterility or bioburden.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates an overall method/device according to an aspect of the present disclosure.

FIG. 2A illustrates a device according to an aspect of the present disclosure.

FIG. 2B illustrates a top view of the device of FIG. 2A.

FIG. 2C illustrates an angled view of the device of FIG. 2A.

FIG. 3 illustrates operating principles of a device of the present disclosure according to an aspect of the present disclosure.

FIG. 4A illustrates a system diagram of an embodiment according to an aspect of the present disclosure.

FIG. 4B illustrates various layers of the of the embodiment as shown in FIG. 4A.

FIG. 4C illustrates a combined mask design of an embodiment according to an aspect of the present disclosure.

FIG. 4D illustrates trap and release efficiency, showing percent of the initial concentration, the percent penetrated, and the percent trapped/released using a design of the present disclosure.

FIG. 5A illustrates a decrease in signal as a cell passes through a single-cell-detection 2-electrode impedance spectroscopy microfluidic chip.

FIG. 5B illustrates a decrease and increase in signal as a cell passes through a single-cell-detection 3-electrode impedance spectroscopy microfluidic chip.

FIG. 5C illustrates original data acquired single with drift.

FIG. 5D illustrates the post processed signal data from FIG. 5C using a baseline correction algorithm for enhanced detection and signal acquisition.

FIGS. 6A-6B illustrate two microfluidic chips where the electrode configuration is in a top-bottom configuration for detecting an impedance change of the whole chamber due to cell growth, or for the trapping of single cells on a substrate, in a channel, or in a porous membrane. FIG. 6A shows the electrodes spaced apart from the porous filter membrane. FIG. 6B shows the electrodes deposited directly on the porous membrane.

FIGS. 7A-7C illustrate an impedance-based sterility and/or bioburden testing device according to an embodiment where a fluidic channel is used as a filter trapping structure to trap and release cells using a tapering fluidic structure and sloped channel design. FIG. 7A shows the microfluidic channels are small enough in their sizes to prevent microbes to move from one chamber to the other. FIG. 7B shows a channel using a sloped two-photon lithography fabrication process to create a gradual decrease in the channel to sub-micron dimensions capable of single cell trapping. FIG. 7C shows an enlarged view of a portion of the embodiment as shown in FIG. 7B.

FIG. 8 illustrates an example of a system for evaluation of sterility or bioburden of a sample according to an embodiment of the present disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Due to the drawbacks of conventional sterility testing, various aspects of the present disclosure pertain to systems and methods to enable rapid, small sample volume, fully automated, small footprint, low power requirements, and in-line sterility testing capabilities. The technology of the present disclosure is generally composed of a microfluidic device that can rapidly concentrate microbial contaminants with minimum loss, incubate concentrated sample material in diverse culture media formulations, followed by rapid single-cell-resolution cell counting (before and after cultivation), to accurately and rapidly determine whether a sample contains viable microorganisms. The microfluidic device utilizes an in-line integrated filtration system to trap and concentrate any contaminants from the solution being measured. Here, the filtration system can be a porous membrane, nanofabricated ceramic sieve, arrays of microfluidic channels, arrays of micro-scale holes, to name a few. The number of contaminants/particles are quantified using impedance detection of the objects, followed by cultivation of the concentrated contaminants under diverse cultivation conditions. Particles/contaminants within this cultivated solution are then enumerated, or sensed, again using impedance detection. Any increase in the number of contaminants/particles detected or increase/decrease in the measured signal indicate the presence of viable microorganisms. This outcome in turn indicates that the tested product is non-sterile. The devices and methods of use can include multiple parallel cultivation chambers each having different cultivation media to test the solution being measured under different cultivation conditions. Impedance-based enumeration of the number of contaminants/particles or impedance-based sensing of the number of contaminants/particles in the solution can be conducted repeatedly for higher accuracy.

Bioburden refers to the number of viable microbes in a given test sample. Similar to sterility testing, bioburden testing is typically performed by taking a percentage of the total reagent or cellular inputs as well as the products to be tested in each manufactured batch. Conventional bioburden testing typically relies on multi-day culture under different growth conditions, such as different growth media, to determine the number of contaminated living microbes in the original solution being tested. These methods are time-consuming, costly, and are not amenable to in-line and/or continuous process monitoring. The systems and methods, as disclosed herein, provide various devices and methods that can enable rapid, small sample volume, fully automated, small footprint, low power requirements, and in-line bioburden testing capabilities. The technology of the present disclosure is generally composed of a microfluidic device that can rapidly concentrate microbial contaminants with minimum loss, incubate concentrated sample material in diverse culture media formulations, followed by rapid single-cell-resolution cell counting (before and after cultivation), to accurately and rapidly determine the number of viable microorganisms in the test sample. The microfluidic device utilizes an in-line integrated membrane filtration system to trap and concentrate any contaminants from the target solution. Here, the filtration system can be a porous membrane, nanofabricated ceramic sieve, arrays of microfluidic channels, arrays of micro-scale holes, to name a few. The initial number of contaminants/particles are quantified using impedance detection of the objects, followed by cultivation of the concentrated contaminants under diverse cultivation conditions. Particles/contaminants within this cultivated solution are then enumerated or sensed again using impedance detection over time. The time-dependent increase in the number of contaminants/particles detected or increase/decrease in the measured signal can be used to enumerate the number of living microorganisms in the original solution. The devices and methods of use can include multiple parallel cultivation chambers each having different cultivation media to test the target solution under different cultivation conditions. Impedance-based enumeration of the number of contaminants/particles or impedance-based sensing of the number of contaminants/particles in the solution can be conducted repeatedly for higher accuracy.

Sterility refers to the non-appearance of viable microorganisms. Therefore, sterility testing is performed to confirm contaminant-free medical devices, tissue materials, and pharma/biopharma materials. If microorganism contamination is identified by sterility testing, the manufacturing process where contamination occurred needs to be pinpointed with the ultimate goal of eliminating all viable microorganisms from the entire manufacturing pipeline. Sterility testing must be conducted for cell banks, cell-based products, genetic vectors, raw materials, and final pharmaceutical offerings, to name a few applications. Sterility testing is also used for testing different preparations, articles, and substances that are required to be made sterile according to the laws set forth by the United States Pharmacopeia (USP), European Pharmacopeia (EP), Japanese Pharmacopeia (JP), and the like. All parenteral preparations made for human usage are subjected to sterility testing to reveal the non-appearance of living microorganisms with tainting ability.

Sterility testing is typically performed by taking a percentage of the total reagent or cellular inputs as well as the products to be tested in each manufactured batch. There are several limitations to current sterility testing methods. First, conventional sterility testing is conducted over a 14-day incubation period as some of the contaminating microorganisms have slow growth rates or require spore germination and growth. Therefore, for thorough determination of the presence of living microorganisms in the sample, which is a source of product contamination, the bio-manufactured product must wait until the testing results are returned (typical lead time is 14-28 days) before it can be released to customers. Thus, the time it takes for sterility testing presents a significant speed bump in the manufacturing of protein and nucleic acid products. Second, the conventional amount of volume needed for testing is substantial (>10 mL at the minimum). This requirement often presents a challenge since many of these therapeutics are precious and costly, and in some instances made for only a very small number of patients or end-users. Third, most current testing methods are not amenable to in-line/continuous process monitoring for sterility. Thus, once a contamination problem has been identified, all batches of therapeutics that were manufactured during that period may have to be discarded (highly costly). The entire manufacturing process must also be examined to identify the point of contamination. These activities result in significant delays in manufacturing and increases in cost. In summary, systems and methods that can provide rapid, small sample volume, fully automated, small footprint, low power requirements, and in-line sterility testing would constitute an ideal solution to overcome these critical bottlenecks.

The technology presented herein can provide whole-lot sterility evaluation as well as in-line continuous sterility monitoring. Compared to conventional approaches, the technology disclosed herein delivers reduction in cost and testing times, respectively, in most cases. The system can also be compact and fully automated. The core technology is a microfluidic integrated membrane filter-based cell concentration and trapping techniques, in conjunction with impedance-based single-cell detection before and after cultivation, to quantify any increase in the number of microorganisms after cultivation, where any increase in number indicate non-sterility of the tested product.

Four challenges and requirements guide this technical solution. First, detecting extremely low concentrations of microbial contaminants (e.g., 1 colony forming unit (CFU)/mL) will require that these contaminants are first highly concentrated with minimum loss before any microbial detection technologies can be applied. The filtration system (e.g., porous membrane, microfluidic channel array, microfabricated hole array)-integrated microfluidic technology provides an ideal solution. Importantly, despite the low cost and simplicity of the proposed method, it can far outperform many other microfluidic cell concentration techniques, such as, dielectrophoresis (DEP), magnetophoresis, and acoustophoresis, to name a few (Table 1).

TABLE 1
Comparison of the cell concentration technology of the present disclosure to other
microfluidic technologies.

| | | | | Technology of the |
|---|---|---|---|---|
| Features | Dielectrophoresis | Magnetophoresis | Acoustophoresis | Present Disclosure |
| No Dead Volume | Yes | Yes | Yes | Yes |
| | Moderate (Relatively | High (If Labeled | Moderate (Small | |
| Efficiency | Weak Force = Slow Flow | with Magnetic | Microbes | High |
| | Speed; Low Conductive | Beads) | Difficult to | |
| | Solution Needed) | | Manipulate) | |
| Microbe Agnostic | No | No | No | Yes |
| Labeling | No | Yes | No | No |
| Requirement | | | | |
| Chip Cost | Low | Low | Moderate | Low |
| Support Instrument | Moderate | Moderate | High (Power | Low |
| Cost | | | Amplifier | |
| | | | Needed) | |

Second, despite many different potential technologies that may be used to detect viable microorganisms, including viability dye-staining methods and label-free vibrational spectroscopy methods (e.g., mid-infrared (MIR), near-IR (NIR), and Raman spectroscopy spectroscopy), determining the viability of microorganisms in extremely small quantities (as low as one cell) is non-trivial. This challenge is amplified by the extremely broad strain diversity of potential microbial contaminants. Measuring cell growth is the ultimate, and generally acceptable, indicator of cell viability, a key reason why USP 71 Sterility Testing requires measurement of microbial growth, and also why a culture-based testing strategy, even though it may take slightly longer, is being pursued.

Third, conventional methods that can measure increasing numbers of cells, such as visual/microscopic observation, flow cytometry, and metabolic activity assays (e.g., measuring pH change from microbial nutrient consumption), require relatively large numbers of cells and/or an initial small number of cells to be grown into 10²-10⁶ cells to produce sufficient signal to be detected. This requirement necessitates either large sample volumes (several tens to hundreds of mL) or long cultivation times (up to several days and/or 15+ cell division cycles). An accurate single-cell-resolution cell measurement method that can enumerate or detect differences in the number of cells both before and after cultivation, as described herein, provides a compelling solution to this challenge (Table 2).

TABLE 2
Comparison of the cell viability/growth detection technology
of the present disclosure to other technologies.

| | | | | | Technology |
|---|---|---|---|---|---|
| | | Metabolic | | | of the |
| | Raman | Activity | Flow | Microscopy/ | Present |
| Features | Spectroscopy | Assays | Cytometry | Optical | Disclosure |
| Single Cell | Yes | No | Yes | Yes | Yes |
| Sensitivity | | | | | |
| Detect Microbial | | | | | |
| Growth/ | No | Yes | No | Yes | Yes |
| Proliferation | | | | | |
| Simplicity | No | Yes | No | Yes | Yes |
| Low Cost | No | Yes | No | No | Yes |
| Label Free/Microbe | | | | | |

Finally, several different single-cell measurement techniques exist. Direct imaging is simple and easy to use, but often requires sophisticated image processing methods, especially to accommodate the broad ranges of potential microbial contaminants. It is also relatively difficult to be used in fully automated and in-line monitoring systems. Flow-through optical detection of single cells is possible, but typically require the cells to be stained, which requires additional sample processing steps, or requires high-speed cameras, which are costly. Impedance spectroscopy-based cell detection, as described herein, is label-free, can detect broad ranges of microorganisms, is high speed, and can be automated. In addition, the sensing instruments can be made compact and portable (Table 2). Taken together, the solution utilizing microfluidic integrated filtration system-based cell concentration and trapping techniques, in conjunction with impedance-based single-cell detection before and after cultivation (measurements at multiple post-cultivation time point as needed), provides a compelling solution that addresses these challenges.

For bioburden measurement, the same background, limitations of current technologies, advantages of the present disclosure, and the like apply. The number of cells or differences in cell number before and cultivation may have to be measured multiple times at different post-cultivation time point so that the data can be used to enumerate the number of living microorganisms in the solution being tested.

The systems, devices, and methods of the present disclosure can be directly developed into a reader/disposable chip combination, providing rapid, sensitive, accurate, and automated assessment of product sterility.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

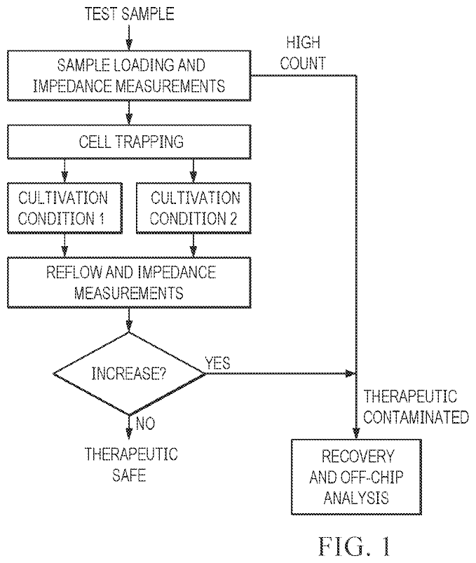

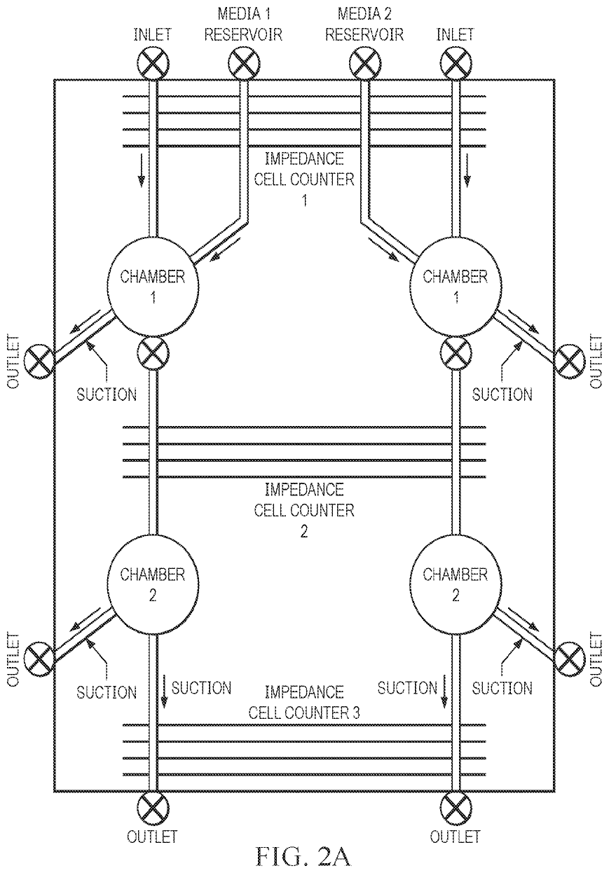

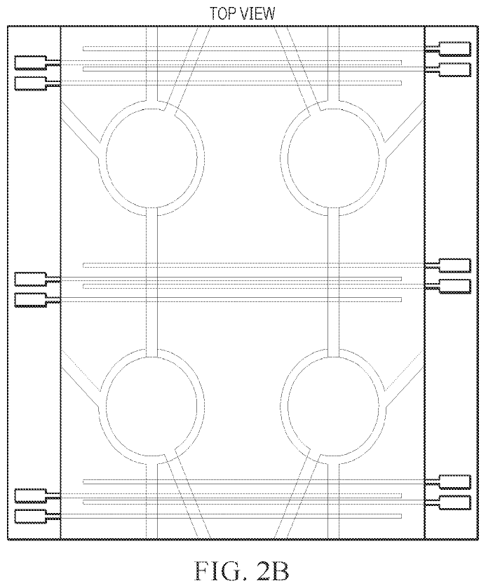

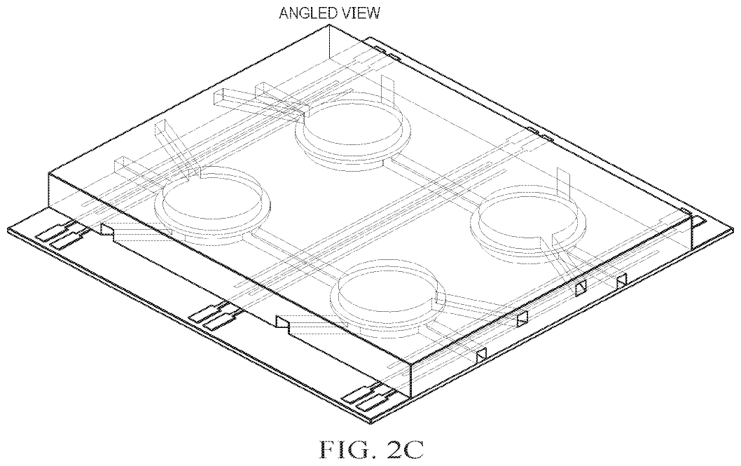

FIG. 1 outlines the overall process flow of the disclosed technology. The system is composed of an integrated cell concentration/filtration system and cell-sensing impedance electrodes. FIGS. 2A-2C show the overall concept of the device and its operating principles according to aspects of the present disclosure. In FIG. 2A, the testing of two different culture media conditions on a single chip (through parallelization) is illustrated. FIGS. 2B-2C show, in general, how the chip can look according to an embodiment of the present disclosure, with FIG. 2B showing the top view, and FIG. 2C showing an angled view. The chip itself can be a compact 3-layer structure (2 cm×2 cm footprint), with a bottom fluidic layer and a top fluidic layer sandwiching a porous membrane filter in the two circular cell trapping/culture chambers.

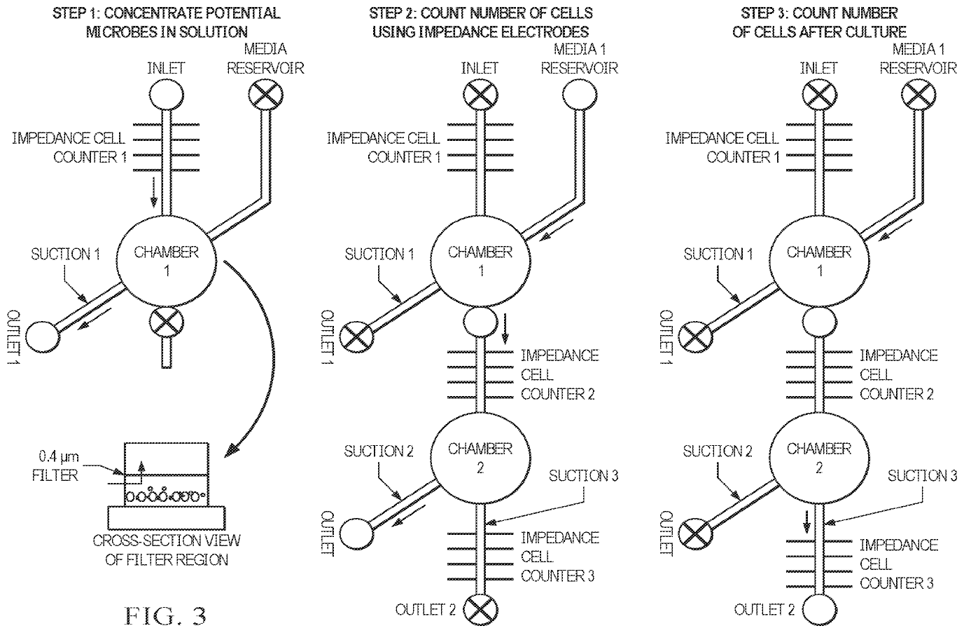

Fluidics Operations. The operation of the device is performed in stepwise fashion. First, as shown in FIG. 3 Step 1, suction pressure is applied to the top part of the circular cell-trapping chamber so that the sample solution flows from the inlet and into suction channel 1 through the integrated membrane filter. Any microbial contaminants and other non-biological particles will thus be trapped in the chamber below the membrane filter. At the same time, the number of cells/objects are counted using impedance cell counter 1. Second, as shown in FIG. 3 Step 2, suction pressure is applied through suction channel 2 so that the culture media flows into the chamber 1 and horizontally moves all trapped cells into chamber 2 where the cells will be trapped using the same principle as shown in Step 1. As the cells move from chamber 1 to chamber 2, the number of cells/objects will be counted using the impedance cell counter 2. Third, the trapped cells are cultivated for a predetermined time as discussed in further detail herein. Finally, as shown in FIG. 3 Step 3, suction pressure is applied from the outlet 2 so that the cultured cells are horizontally released and flow through the impedance cell counter 3, resulting in post-culture cell counting. If the number of counted objects increases, it is deemed that living microorganisms were detected and cultivated in the chip, indicating that the test sample is contaminated. If the number of counted objects remains the same, it is determined that the concentrated objects were non-viable or non-cellular particles, and thus the tested sample determined to be sterile.

Importantly, the final sample can be recovered from the outlet channel. Thus, if the sample is determined to be contaminated, 16S sequence analysis can be conducted on this collected sample to determine the identity of the microbiological contaminants. Several pneumatically actuated pinch valves can control the flow during the various operations. The opening and closing of these valves are illustrated in FIG. 3 as an open circle and an X-marked circle, respectively.

Impedance-Based Single-Cell Sensing. The initial impedance electrode design can be a planar and parallel electrode design (e.g., a 2-electrode-pair design), where an electrode-to-electrode gap of 5-20 micrometers is utilized. The initial microchannel height is 10 micrometers, which can be further optimized in the 10-20 micrometer range. The electrode design can be also a 3-electrode-pair design for improved sensing capability.

Parallel Operations. In the case of two parallel channels for testing two different media conditions (e.g., embodiments shown in FIGS. 2A-2C), the suction channels can be shared, as well as the opening and closing of the valves. In addition, a single impedance electrode pair spanning both parallel microchannels can be utilized to count the number of cells passing by (e.g., embodiments shown in FIGS. 2A-2C). This configuration minimizes the electrical interconnects needed in the chip. Although a two parallel device design that can test two different cultivation condition is shown here, the number of parallel channels and devices on the same substrate is not limited to two, and can be more than two.

For storage of the cultivation media and various buffer solution, an on-chip reservoir pre-filled with the respective culture media or buffer can be utilized (e.g., media 1 reservoir of FIG. 2A), from which the media or buffer can be drawn into the cell culture chamber (e.g., chamber 2 of FIG. 2A), together with the trapped cells in the preceding chamber (e.g., chamber 1 of FIG. 2A). Since the microfluidic device can be made in flexible polydimethylsiloxane (PDMS) the media can be loaded into the media reservoir that is already sealed by injecting the media using a needle-point syringe. Since PDMS is a rubber-like material, after taking out the syringe needle, it will be self-sealed. Even though the example device material used is PDMS, the material of the device is not limited to PDMS, and rather can be any combination of commonly utilized microfabrication/microfluidic materials, including other plastics (e.g., polycarbonate), silicon, glass, to name a few.

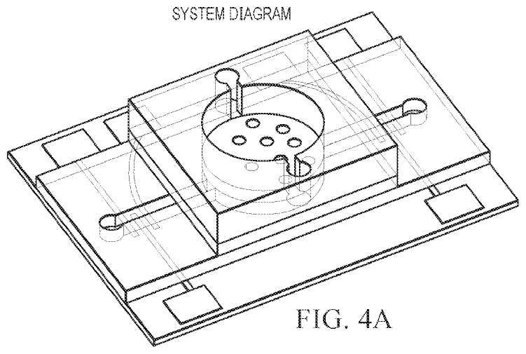

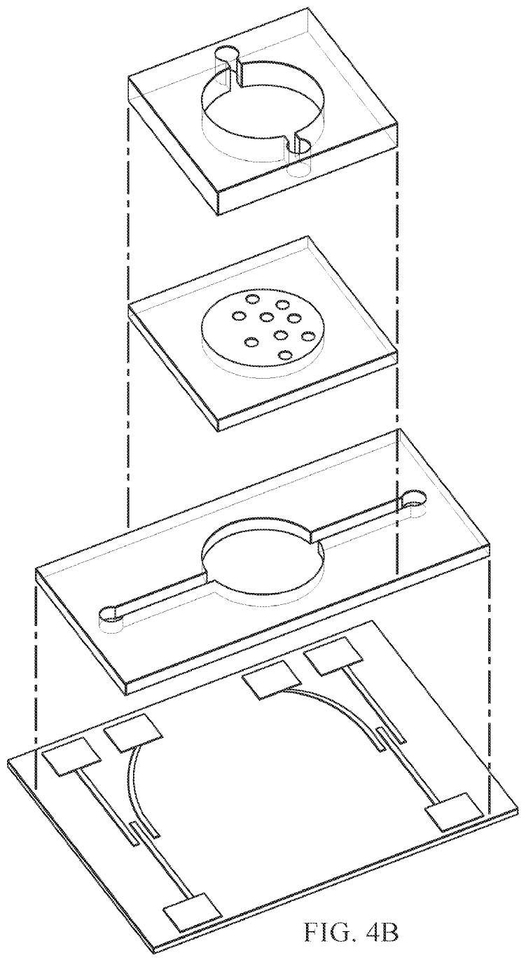

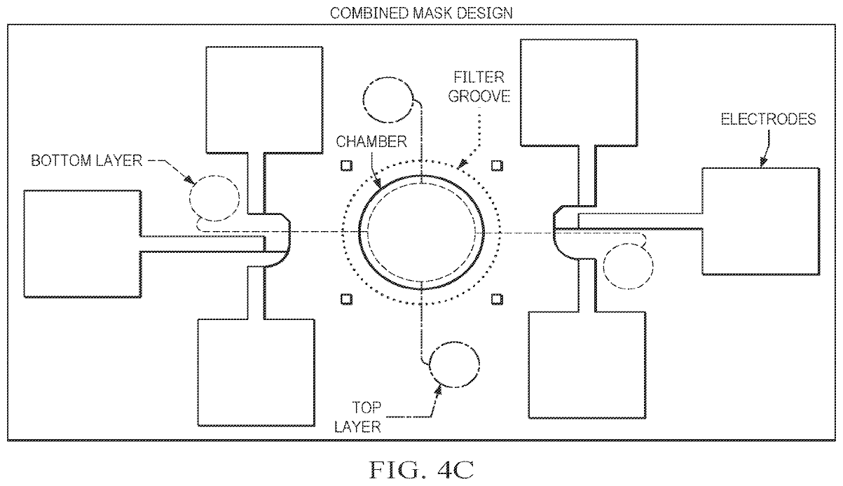

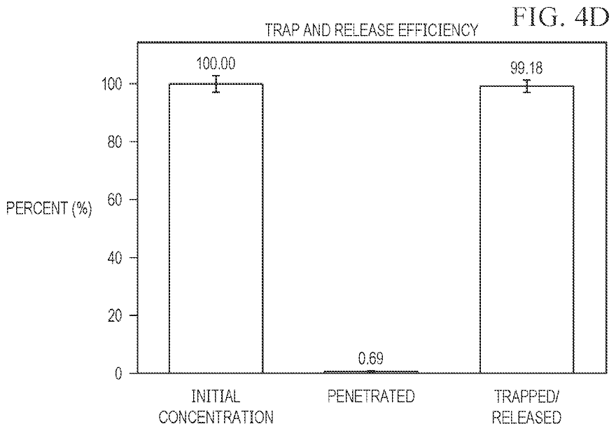

Integrated Microfluidic Membrane-Based Cell Concentration, Trapping, and Solution Exchange. Porous membrane filters having different pore sizes have been integrated into the microfluidic channels for high-efficiency trapping of cells, solution exchange while trapping the cells, and cell washing. FIGS. 4A-4D show a design concept and data, where a membrane filter integrated into a microfluidic channel allowed trapping of a large numbers of cells, solution exchange while holding onto the cells, and further manipulation of the trapped cells to a different location with near zero loss. FIG. 4A shows a system diagram of an embodiment according to an aspect of the present disclosure, while FIG. 4B shows various layers of the of the embodiment as shown in FIG. 4A. FIG. 4C shows a combined mask design of an embodiment according to an aspect of the present disclosure. Trap and release efficiency are demonstrated in FIG. 4D, which shows the percent of the initial concentration, the percent penetrated, and the percent trapped/released.

This concept has been integrated as a fully automated complex cell manipulation system and its operation demonstrated using cells of two different size (E. coli and mammalian cells). This basic principle is at the core of the disclosed technology in concentrating cells from test samples, loading culture media into the chamber while holding onto the concentrated cells and culturing them, releasing and counting the replicated cells at a single-cell resolution, as discussed in further detail herein.

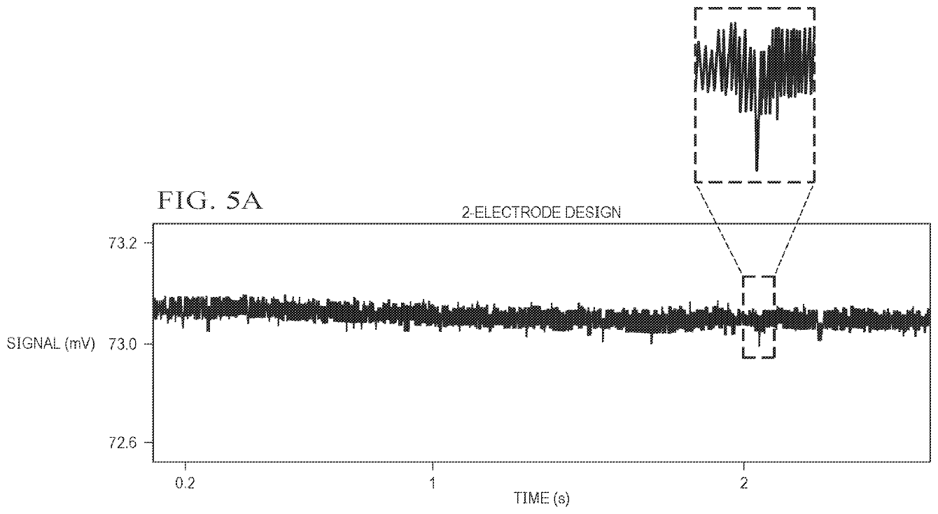

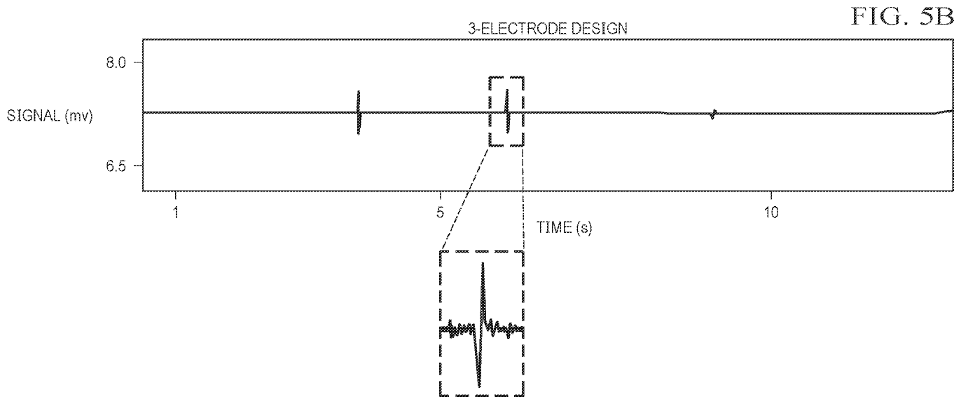

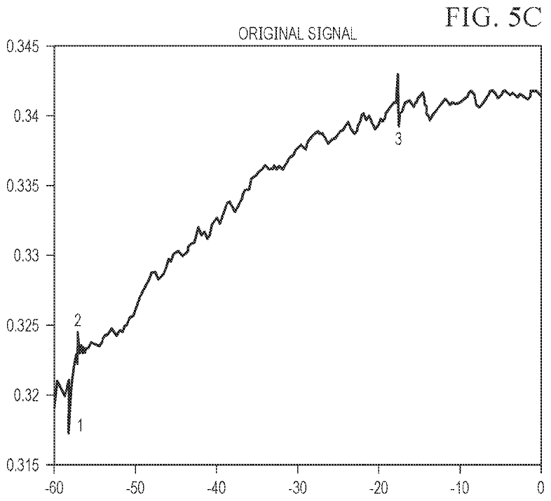

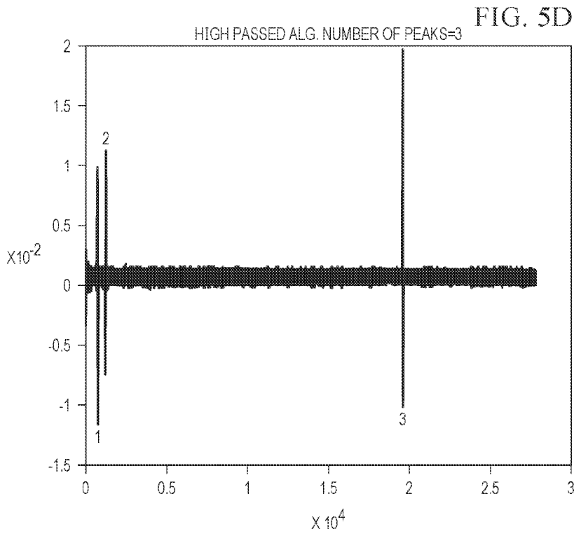

Single-Cell-Resolution Impedance Sensing of Single Cells. Impedance-based cell detection and characterization can be utilized. FIGS. 5A-5D show data relating to embodiments where a microfluidic channel having impedance-detection planar electrodes on the bottom of the microchannel allows detection of single cells passing through the microfluidic channel. FIG. 5A shows a decrease in signal as a cell passes through a single-cell-detection 2-electrode impedance spectroscopy microfluidic chip, while FIG. 5B shows a decrease and increase in signal as a cell passes through a single-cell-detection 3-electrode impedance spectroscopy microfluidic chip. FIG. 5C shows the original acquired single with drift, while the post processed signal using a baseline correction algorithm for enhanced detection and signal acquisition is shown in FIG. 5D. Typical detection speeds are 400 cells/sec, providing sufficient detection speed for rapid cell counting. The detection speed is not limited to 400 cells/sec, and can be higher if a higher flow speed is utilized.

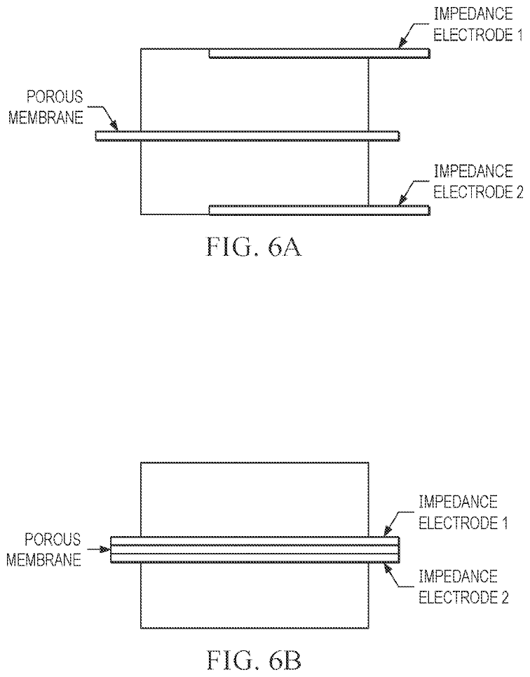

FIG. 6A shows an embodiment of the microfluidic chip electrodes where electrode pairs are placed on the top and bottom of a channel, device, substrate or pore to detect cells as they pass, are trapped, or fill up the chamber due to growth. FIG. 6B shows an additional embodiment of this classification may be a porous trapping designed using a microfluidic approach where the electrodes are on the membrane where cells are being trapped. Such embodiments are discussed in further detail below.

Variations of the Impedance-Based Sterility Testing Device Design. FIGS. 6A-6B show two variations. FIG. 6A shows the electrodes spaced apart from the porous filter membrane, while FIG. 6B shows the electrodes deposited directly on the porous membrane. In these designs, the two top/bottom chambers separated by a porous membrane, which forms the cell-concentration/trapping chamber as initially discussed herein, has an impedance-sensing electrode within each chamber. The electrical impedance measured between the top and bottom chamber indicate the degree of blockage of the porous membrane by the microbes. If the solution being tested is not sterile and microbes grow in number in this cultivation chamber, more microbes will block the pour and thus result in changes in the electrical impedance between the two chambers. In these designs, it may be needed to culture the microbes on the top chamber so that as the microbes naturally settle down on top of the porous membrane, a more sensitive detection may be possible. It may also be needed to apply suction pressure from the chamber holding the microbes (in this case the top chamber) towards the empty chamber (in this case the bottom chamber) to further improve microbes coming in contact with the porous chamber. The surface area of the porous membrane area may be minimized to maximize the sensitivity of microbial detection. A change in the impedance signal means that the sample contains growing microbes, thus the target solution is not sterile (have living microorganisms). In some embodiments, as shown in FIG. 6B, the porous filter membrane has the electrodes deposited directly on the membrane and detect single cells or the increase of cells being trapped in individual pores, at particular sites or locations, or in a bulk format similar to that in FIG. 6A.

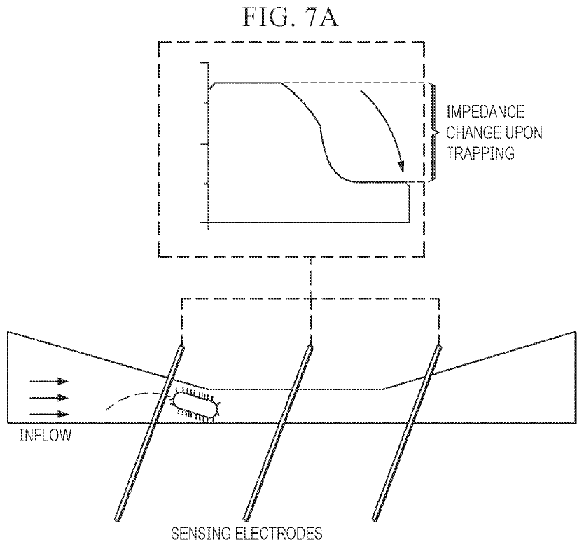

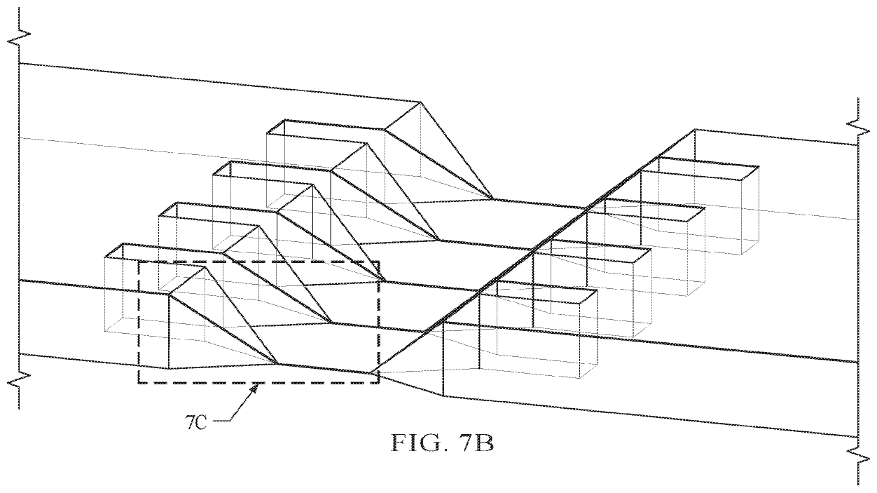

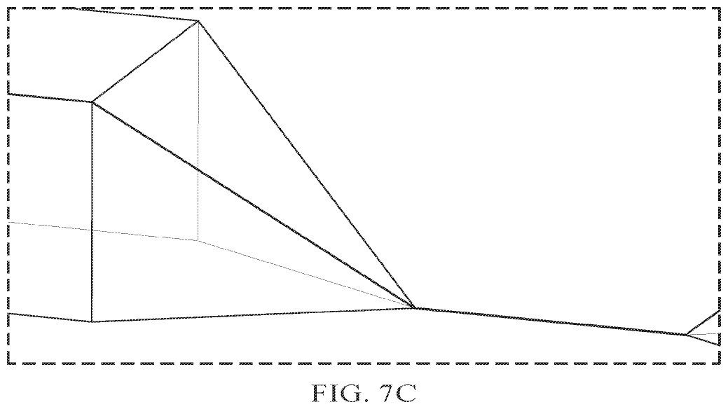

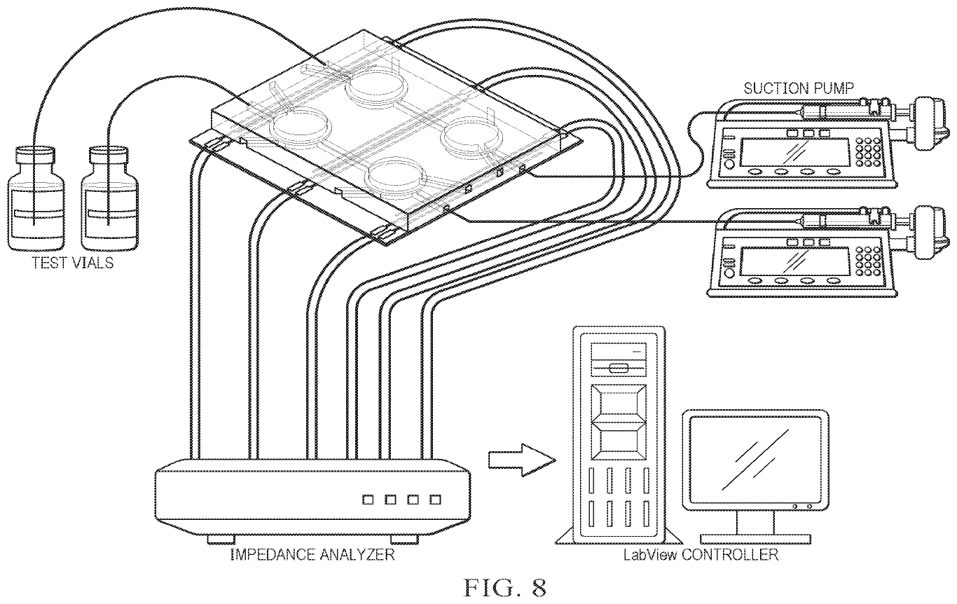

An additional design, illustrated in FIGS. 7A-7C, utilizes the same concept but is composed of two horizontals microbial cultivation chambers connected through arrays of microfluidic channels. These microfluidic channels are small enough in their sizes to prevent microbes to move from one chamber to the other (FIG. 7A). The channel uses a sloped two-photon lithography fabrication process to create a gradual decrease in the channel to sub-micron dimensions capable of single cell trapping (FIG. 7B, with FIG. 7C showing an enlarged view of a portion of the embodiment as shown in FIG. 7B). The printed master-mold structure has chambers and a stair-step like sloping design with nanometer scale resolution. Each chamber has an impedance sensing electrode or electrodes that span all channels. Applying flow or suction from one side of the chamber to the other side of the chamber will result in the microbes to block a percentage of the microfluidic channels. Such blockage will result in changes in the electrical impedance between the two chambers. If the number of microbes increase due to microbial growth in the cultivation chamber, more significant changes in electrical impedance signal will occur, allowing the sensing of the number of microbes in one chamber. A change means that the sample contains growing microbes, thus the target solution is not sterile (have living microorganisms). Repeated measurement can be conducted by releasing the microbes blocking (either fully or partially) the microfluidic channels back to the chamber it came from, cultivated, and re-measured by repeating the process described above.

In another method of obtaining impedance signal, the microfluidic channels can be designed to allow microbes to flow through the microfluidic channels. The microbes flowing through the microfluidic channels will result in changes in impedance signal. As the microbes are cultured and increase in their numbers, this impedance signal will change. A change means that the sample contains growing microbes, thus the target solution is not sterile (have living microorganisms).

FIG. 8 shows an example of a system for evaluation of sterility or bioburden of a sample according to an embodiment of the present disclosure. As shown in FIG. 8, test vials are connected to a microfluidic device such that flow of a sample through the microfluidic device is controlled via suction pumps. An impedance analyze is connected to integrated impedance sensing electrode arrays on the microfluidic device, and corresponding data is sent to a controller for analysis and/or automation (e.g., a LavView controller).

It should be noted that all designs of the present disclosure used for sterility testing can also be utilized for bioburden testing. The only difference is that from the impedance signal change, the number of living microorganisms in the original sample has to be calculated.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A method for evaluation of sterility in a solution using impedance sensing, the method comprising:
filtering and concentrating microbial or other contaminants in the solution;
counting and enumerating an initial number of concentrated microbial or other contaminants, wherein the presence of enumerated microbial or other contaminants is indicative of a potential microbial contaminant in the solution;
cultivating each microbial or other contaminant, independently or together, for a variable period of time in one or more culture media, wherein the cultivating increases the number of each microbial or other contaminant;
detecting each cultivated microbial or other contaminant;
counting and enumerating the number of each detected cultivated microbial or other contaminant; and
comparing the number of each detected cultivated microbial or other contaminant with the initial number of concentrated microbial or other contaminants in the solution, wherein an increase in the number of each detected cultivated microbial or other contaminant is indicative of the solution being non-sterile, and wherein the microbial or other contaminants are counted using a label-free impedance sensing function.

2. The method of claim 1, wherein the concentrating comprises a method selected from the group consisting of physical filtration methods, physical filtration utilizing porous membrane filters, microfluidic channel cell trapping structures, dielectrophoretic force, acoustophoretic force, concentrating methods, and combinations thereof.

3-4. (canceled)

5. The method of claim 1, wherein at least a subset of the steps is conducted using a label-free approach.

6. The method of claim 1, wherein the evaluation of sterility is conducted in in at least one of less than 5 days, 3 days, 1 day, 12 hours, 6 hours, 3 hours, 1 hour, 30 minutes, 1 minute, or combinations thereof.

7-9. (canceled)

10. The method of claim 1, wherein the evaluation of sterility is combined with at least one of other methods for detection of microbial viability, microbial metabolism, or combinations thereof to provide complimentary information on whether the solution contains any living microorganisms.

11-14. (canceled)

15. A method for evaluation of bioburden in a solution, the method comprising:
filtering and concentrating microbial or other contaminants in the solution;
counting and enumerating an initial number of concentrated microbial or other contaminants, wherein the presence of enumerated microbial or other contaminants is indicative of a potential microbial contaminant in the solution;
cultivating each microbial or other contaminant independently in one or more culture media, wherein the cultivating increases the number of each microbial or other contaminant;
counting and enumerating a number of each detected cultivated microbial or other contaminant; and
comparing the number of each detected cultivated microbial or other contaminant with the initial number of concentrated microbial or other contaminants in the solution, and wherein the microbial or other contaminants are counted using a single-cell-resolution label-free impedance sensing function.

16-18. (canceled)

19. The method of claim 15, wherein at least a subset of the steps is conducted using a label-free approach.

20-21. (canceled)

22. A device for evaluation of sterility or bioburden, the device comprising:
a filtration and concentration microfluidic apparatus to filter and concentrate microbial contaminants from a solution using a filtration structure;
wherein the filtration structure comprises a porous membrane filter;
wherein a pore size in the membrane filter is similar or smaller than a size of the microbial contaminants such that particles or microorganisms can be trapped by the filtration structure;
a microfluidic channel comprising at least one integrated impedance sensing electrode array to detect single cells passing through an electrode of the electrode array for label-free single-cell-resolution flow through counting of the particles or microorganisms;
a cultivation microfluidic apparatus, wherein filtered, concentrated, and enumerated particles or microorganisms are operable to be moved into one or more cultivation chambers comprising one or more different microbial cultivation media to allow growth of microorganisms for varying durations;
at least one of a series of microfluidic valves placed in each microfluidic channel operable to control closing and opening of each microfluidic channel, or a series of valving mechanisms to control flow of fluid on-chip;
wherein valving mechanisms have a location selected from the group consisting of on-chip, off-chip, between components, integrated at varying locations between tubing interconnects, and combinations thereof; and
a software interface operable to count differences in a number of detected contaminant particles before and after cultivation, wherein any increase in number of contaminant particles indicate the solution is non-sterile.

23-24. (canceled)

25. The device of claim 22, wherein the filtration and concentration microfluidic apparatus comprises:
a bottom microfluidic channel through which microbial contaminants flow into a porous membrane filter region;
a first microfluidic chamber comprising a porous membrane filter as the top portion of the first microfluidic chamber;
wherein the porous membrane filter comprises pore sizes similar or smaller than microbial contaminants to be concentrated;
a top microfluidic channel and chamber, wherein suction pressure can be applied such that the flow is from the bottom microfluidic channel, through the porous membrane filter, and into the top microfluidic channel such that all microbial contaminants are trapped in the bottom microfluidic channel;
a media reservoir in fluid communication with the top microfluidic chamber through a microfluidic channel;
an outlet channel, wherein suction pressure can be applied so that flow is created from a test solution inlet to an outlet;
wherein a pore size of the porous membrane filter can be selected with different sizes to maximize trapping efficiency while further maximizing flow rate through the porous membrane filter;
at least one of a series of microfluidic valves that are placed in each microfluidic channel to control closing and opening of the microfluidic channels, or a series of valving mechanisms to control flow of fluid on-chip; and
wherein valving mechanisms have a location selected from the group consisting of on-chip, off-chip, between components, integrated at varying locations between tubing interconnects, and combinations thereof.

26. The device of claim 22, comprising an impedance counting electrode disposed between an inlet and the filtration and concentration microfluidic apparatus operable to count the number of incoming particle contaminants.

27. The device of claim 22, comprising:
a microchannel with integrated impedance electrodes that connects the filtration and concentration microfluidic apparatus to the cultivation microfluidic apparatus;
a cultivation chamber in a lower portion of the cultivation chamber, wherein microbial and other contaminants can be trapped by the porous membrane filter placed on top of the cultivation chamber;
wherein the porous membrane filter that covers the ceiling of the cultivation chamber;
an upper microfluidic chamber placed on top of the porous membrane filter;
a second outlet in fluid communication with the upper microfluidic chamber of the cultivation chamber such that suction pressure can be applied;
wherein applying the suction pressure through the second outlet results in media from a first media reservoir to flow into the lower portion of the cultivation chamber, take any concentrated microbial and other contaminants, and flow into the cultivation chamber;
wherein flow moves through the porous membrane filter allowing continuous flow while all concentrated microbial and other contaminants remain in the cultivation chamber thereby moving all microbial and other contaminants from the filtration and concentration microfluidic apparatus to the cultivation chamber and resuspending the microbial and other contaminants into the microbial cultivation media;
a microfluidic channel in fluid communication with the cultivation chamber and to an outlet, wherein impedance sensing electrodes are integrated into the microfluidic channel;
at least one of a series of microfluidic valves that are placed in all microfluidic channels to control closing and opening of all microfluidic channels, or a series of valving mechanisms to control the flow of fluid on-chip;
wherein valving mechanisms have a location selected from the group consisting of on-chip, off-chip, between components, integrated at varying locations between tubing interconnects, and combinations thereof; and
a device component operable to be automated by at least one of a controller or an automation system comprising components that are optically transparent thereby allowing simultaneous interrogation of microbial contaminants using optical and impedance directed approaches.

28-29. (canceled)

30. The device of claim 22, wherein the at least one integrated impedance sensing electrode array comprises:
a planar electrode design having one pair of electrodes close together and placed on a bottom portion of the microfluidic channel to detect an object passing above the planar electrode through changes in impedance at various applied voltages and frequencies;
wherein the planar electrode design that has two or more electrodes in an interdigitated form;
a top-bottom electrode design, wherein one or more electrodes are placed on the surface of the microfluidic channel and one or more electrodes are placed on the ceiling of the microfluidic channel;
wherein the location of the electrodes have an orientation selected from the group consisting of stacked directly on top of each other and stacked at some distance apart from each other;
a three-dimensional electrode design, wherein one or more electrodes are on one side of the microfluidic channel and another electrode is on an opposite side of the microfluidic channel; and
wherein the electrodes comprise at least one of metals, liquid metals, conductive solutions or materials, or combinations thereof;

31. The device of claim 22, wherein the filtration and concentration microfluidic apparatus comprises:
an inline filter placed perpendicular to a horizontal flow inside the microfluidic channel;
wherein the inline filter has pore sizes that are similar or smaller than a target contaminant size;
wherein the inline filter can be microfabricated directly within the microfluidic channel;
wherein the inline filter can be physically assembled between two microfluidic channels;
a second microfluidic channel placed in front of the inline filter and placed perpendicular to main flow microfluidic channel;
wherein a cross flow can move concentrated microbial and other contaminants to a downstream impedance sensing region;
at least one of a series of microfluidic valves that are placed in all microfluidic channels to control closing and opening of all microfluidic channels, or a series of valving mechanisms to control the flow of fluid on-chip; and
wherein valving mechanisms have a location selected from the group consisting of on-chip, off-chip, between components, integrated at varying locations between tubing interconnects, and combinations thereof.

32. The device of claim 22, wherein two or more apparatuses are placed in parallel to test microbial cultivation under two or more cultivation conditions in parallel;
wherein impedance sensing electrodes are integrated into two or more microfluidic channels so that cells flowing in the two or more microfluidic channels can be counted using only a single impedance sensing apparatus; and
wherein a single microvalve controller can control the two or more apparatuses simultaneously.

33. The device of claim 22, wherein cultivation media is pre-loaded into a cultivation chamber, and wherein a media reservoir is replaced with a buffer reservoir.

34. The device of claim 22, comprising a device where impedance is controlled by at least one of an automated controller, an automated system, or combinations thereof.

35-36. (canceled)
